(19) United States
(12) Patent Application Publication
(10) Pub. No.: US 2022/0160028 A1
(43) Pub. Date: May 26, 2022

(54) ROCK INSERT FOR SMOKING OR VAPING

(71) Applicant: Clay Houser, Denver, CO (US)

(72) Inventor: Clay Houser, Denver, CO (US)

(21) Appl. No.: 17/529,412

(22) Filed: Nov. 18, 2021

Related U.S. Application Data
(60) Provisional application No. 63/117,209, filed on Nov. 23, 2020.

(51) Int. Cl. A24F 9/00; A24F 42/10; A24F 42/60

(52) U.S. Cl. CPC A24F 9/00; A61M 11/041; A24F 42/60; A24F 42/10

(57) ABSTRACT

A rock insert for use with smoking or vaping paraphernalia such as a smoking pipe. The rock insert includes a porous body having an interior including first and second interior regions for holding smokable or vaping material, and a closed bottom. The porous body includes a plurality of pores, or vesicles having different shapes and dimensions that are randomly formed to absorb heat generated by an external heating source. The porous body effectively absorbs heat, and gradually and evenly distributes heat in a controlled manner to the smokable or vaping material to produce a gaseous vapor for human consumption. The rock insert is compatible for use with heat sensitive concentrates that can be heated slowly without burning or combusting quickly. In one embodiment, the rock insert includes a first interior formed in one end of the porous body, and a second interior formed in another end of the porous body, opposite the one end, to provide alternative interiors for holding smokable and/or vaping materials therein.

### CROSS-REFERENCE

This application claims the benefit of U.S. Provisional Patent Application No. 63/117,209, filed on Nov. 23, 2020, titled, “Rock Insert for Vaping”, the entire contents of which is hereby incorporated by reference in its entirety.

### FIELD OF INVENTION

The present invention relates to accessories or devices associated with smoking or vaping practices, and more particularly, to a rock insert adapted for use with smoking and/or vaping paraphernalia and devices. The rock insert includes a porous body that absorbs heat generated by an external heating source for gradually heating smokable or vaping material held within an interior of the porous body to produce a gaseous vapor for human consumption.

### BACKGROUND OF THE INVENTION

Smoking or vaping devices have been developed over the years for individuals to enjoy various brands of smokable or vaping materials. A wide selection of smokable or vaping materials has been introduced to solicit the interest of users who wish to experience alternative choices of flavors, strengths and aroma. Such smokable or vaping materials may include for example, different types of flavored or essential oils, tobaccos, or herbal plants or extracts. A traditional device typically used for smoking a plant-based product such as tobacco includes the well-known tobacco pipe. The tobacco pipe generally includes a pipe bowl joined to a stem having a delivery channel and a mouth piece for inhaling a gaseous vapor that is produced when igniting smokable material with an external heat source such as a butane lighter, or match. Over time, various efforts have been made to improve on the style, use and operation of conventional tobacco pipes. For example, a number of different filters have been designed to filter the inhaled gaseous vapor, devices have been engineered to enhance the circulation of air within the pipe bowl to increase combustion of the smokable material, and insulation materials have been introduced to shield a user's hand from excess heat during use.

In an effort to attract consumers, overcome certain unpleasantries associated with traditional smoking, and economize on the construction and use of conventional smoking devices, manufactures have developed a variety of electronic vaping devices. Electronic vaping devices provide a compact, self-operating, all-in-one device that does not require an external heating source as needed with traditional smoking pipes, and accommodates use with a variety of liquid concentrates having different flavors, or consistencies. Electronic vaping devices typically include an internal heating element electrically coupled to an enclosed power source such as a disposable or rechargeable battery, via a control switch, and a small chamber for removably receiving a receptacle filled with a liquid concentrate. Upon activating the control switch, the internal heating element is energized to heat the liquid concentrate through conduction of thermal energy elevating the temperature of the liquid concentrate to produce a gaseous vapor for inhaled, human consumption.

Although traditional smoking pipes and electronic vaping devices offer particular advantages, such devices also have certain drawbacks. For example, smoking pipes are tailored for use with smokable materials that comprise plant-based substances and cannot be employed for use with liquid concentrates. Electronic vaping devices typically include heating elements that are specifically designed to operate at certain temperatures often producing excessive, high intensity heat in a very short period of time. Heat sensitive concentrates vaporize quickly when exposed to high temperatures thus preventing the vapor from being captured and delivered in a timely manner for human consumption. Concentrates that are exposed to high temperatures or prolonged heat often produces a vapor having a bitter taste, a lower potency level, and burns quickly. Another drawback is that many electronic vaping devices are tailored for use with liquid concentrates and are not compatible for use with solid or semi-solid smokable materials thus limiting the choice of smoking materials for users.

Accordingly, there is an established need for a solution to at least one of the aforementioned problems. There remains a need for a smoking or vaping accessory that includes a rock insert adapted for use with traditional, existing smoking and/or vaping paraphernalia such as a smoking pipe for heating smokable or vaping material in a controlled manner to produce a gaseous vapor for human consumption without the risk of burning the smokable material too quickly during use. There also remains a need for a rock insert that is small, compact, easily transportable, and is constructed from an abundance of natural resources.

### SUMMARY OF THE INVENTION

An objective of the present invention is to provide a rock insert that is adapted for use with a variety of different smoking or vaping paraphernalia and devices. The rock insert is constructed from a natural resource such as volcanic rock having advantageous heating properties that effectively absorbs heat when exposed to an external heating source and gradually delivers the absorbed heat to smokable or vaping material provided within the rock insert to produce a gaseous vapor without burning or combusting the smokable or vaping material.

One embodiment of present invention provides a rock insert for use with smoking and/or vaping materials, said rock insert comprising: a porous body including an interior extending partially within a central region of the porous body, a top opening in communication with the interior, and a closed bottom, the interior including a first interior region having a first interior sidewall integral with an annular base, annular ledge, or circular ledge, and a second interior region formed through the annular base, annular ledge, or circular ledge and coincident with the first interior region, the second interior region including a second interior sidewall having a top peripheral edge and a closed base, the second interior region being smaller in size than the first interior region, and wherein the porous body is heatable by an external heating source to gradually distribute heat to a smokable or vaping material deposited within the interior to produce a gaseous vapor for human consumption.

In one aspect, the porous body comprises a cylindrical porous body having an outer diameter and height. The first interior region comprises a first diameter, and the second interior region comprises a second diameter, where the second diameter is smaller than the first diameter.

In one aspect, the porous body comprises a microporous, macroporous, cryptoporous, ultramicroporous, or mesoporous material. The porous body comprises any of volcanic rock, lava, scoria, basalt, andesite, rhyolite, dacite, latite, pumice, pyroclastic rock, sandstone, siltstone, mudstone, shale, granite, Trachytic obsidian, Trachyte, Andesitic glass, Syenite, Granodiorite, Quartz diorite, Rhyolitic obsidian, various tuffite, clay, glass, firerock, limestone, or any combination thereof. In another embodiment the porous body is constructed from material that regulates heat and absorbs liquid that is gradually heated. The porous body includes a plurality of pores and/or vesicles having different sizes, shapes, and depths that are randomly formed within the body of the rock insert. The plurality of pores and/or vesicles do not extend completely through the porous body. Each of the plurality of pores and/or vesicles penetrate partially within the porous body of the rock insert.

In another aspect, the rock insert further includes a porous cap that is removably attachable to the top opening of the porous body for closing the interior. The porous cap comprises a plurality of pores and/or vesicles similar to the porous body.

In still another aspect, the rock insert further includes an alternate interior that is formed within the closed bottom, and includes an alternative sidewall integral with an alternate closed base where the alternate interior is formed opposite the interior. Smoking or vaping material of varying consistencies, or densities may be held in either the interior or alternate interior during use.

In another aspect, the rock insert may include engagement or friction features formed integral with, or separately attached to, the outer surface of the porous body to help removably retain the rock insert within a designated recess, opening or orifice of a smoking or vaping paraphernalia. The engagement features may include for example, one or more spirals, ridges, protrusions or nubs, band, or O-ring having heat resistant properties.

These and other objects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is a top, perspective view of a rock insert including a porous body including an interior extending within the porous body for holding a smokable or vaping material, a top opening in communication with the interior, and a closed bottom, in accordance with an embodiment of the present invention;

FIG. 2 is a bottom, perspective view of the rock insert of FIG. 1, showing the porous body including the closed bottom having a rounded contour;

FIG. 3 is a top view of the rock insert of FIG. 1, showing the top opening of the interior including a first interior region in communication with a second interior region adjoined to the closed bottom, the first interior region including a first diameter, and the second interior region including a second diameter that is smaller than the first diameter;

FIG. 4 is a perspective, cross-sectional view of the rock insert of FIG. 1 taken along lines A′-A′ in FIG. 3, showing the first interior region including a first sidewall integral with a base, and the second interior region extending through the base defining a second sidewall and a closed base;

FIG. 5 is a perspective view of the rock insert of FIG. 1, and a partial view of a vaping or smoking paraphernalia comprising a smoking pipe including a pipe bowl joined to a stem including a delivery channel shown in broken lines;

FIG. 6 is a top view of the rock insert of FIG. 1, and the pipe bowl of the smoking pipe of FIG. 5, showing the rock insert removably inserted within the pipe bowl providing viewable access to the interior of the rock insert;

FIG. 7 is a perspective, cross-sectional view of the rock insert seated within the pipe bowl taken along lines B′-B′ of FIG. 6, showing an open flame directed towards the upper region of the rock insert allowing the porous body to absorb heat to gradually heat smokable or vaping material provided within the interior of the porous body producing a gaseous vapor delivered through the stem of the smoking pipe for inhaled consumption by users;

FIG. 8 is a perspective view of the rock insert of FIG. 1, showing a cap removably attachable to the top opening of the porous body for shielding smokable or vaping material within the interior of the rock insert;

FIG. 9 is a bottom, perspective view of a rock insert showing a porous body including an interior extending within the porous body for holding a smokable or vaping material, a top opening in communication with the interior, and a bottom, and an alternate interior formed within the bottom for holding smokable or vaping material, in accordance with an alternative embodiment of the present invention; and

FIG. 10 is a side, perspective view of a rock insert showing a porous body including a first interior formed in one end of the porous body, and a second interior formed in an opposite end of the porous body, each first and second interior adapted for holding smokable or vaping material, in accordance with another embodiment of the present invention.

### DETAILED DESCRIPTION

The following detailed description is merely exemplary in nature and is not intended to limit the described embodiments or the application and uses of the described embodiments. Terms and phrases used in this document, and variations thereof, unless otherwise expressly stated, should be construed as open ended as opposed to limiting. The term “including” should be read as meaning “including, without limitation” or the like; the terms “illustrative”, “exemplary”, or “example” is used to provide exemplary instances or illustrations of the item in discussion, and not an exhaustive or limiting list thereof, or not construed as preferred or advantageous over other implementations; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Furthermore, the use of plurals can also refer to the singular, including without limitation when a term refers to one or more of a particular item; likewise, the use of a singular term can also include the plural, unless the context dictates otherwise.

All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is understood that the specific devices and processes illustrated in the attached drawings, and described in the following specification, are simply exemplary embodiments of the inventive concepts defined in the appended claims. Hence, specific dimensions, exploded views, and other physical characteristics relating to the embodiments disclosed herein are not limiting, unless the claims expressly state otherwise.

The term, “smokable or vaping material” as used herein, refers to any compound, mixture, blend, concentrate, agent, powder, solid, semi-solid, wax, oil, gel, or liquid, comprising any of, tobacco, nicotine, herbs including, for example, mullein, skullcap, coltsfoot, mugwort, uva-ursi, mint, and sage, other plants or flowers including California poppy, hops, honeyweed, lavender, lily, passion flower, wireweed, natural or herbal teas, flavored oils, essential oils, blends of essential oils, infused waxes, hemp, extracts, Cannabis sativa, marijuana, Cannabis indica, or cannabidiol (CBD), or vaporizing flavors. The smoking or vaping material may also include medical agents or medical concentrates for use in medical applications associated with pulmonary function, nasal or sinus congestion, oral hygiene, stress management, depression, or pain management.

The term, “porous” as used herein includes a wide range of porous materials, including but not limited to, cryptoporous, ultramicroporous, microporous, mesoporous, macroporous, or having other pore sizes, but it also includes materials having a combination of different pore sizes. Porous also refers to a plurality of pores and/or vesicles having different sizes, shapes, and depths that are randomly formed within the body of the rock insert. The plurality of pores and/or vesicles do not extend completely through the porous body. Each of the plurality of pores and/or vesicles penetrate partially within the porous body of the rock insert, and any number of pores and/or vesicles may interconnect with each other.

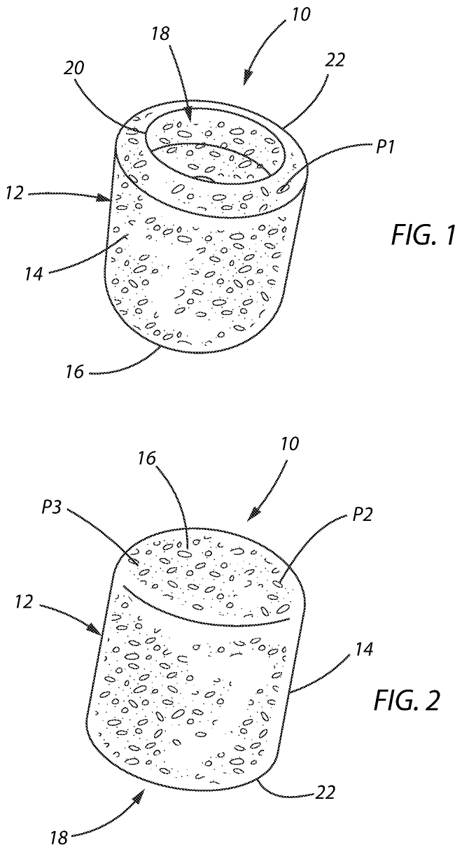

Referring now to the figures wherein like elements are represented by like numerals throughout, there are shown in FIGS. 1 and 2, a top and bottom perspective view, respectively, of a rock insert 10 adapted for use with a variety of traditional or specifically constructed vaping and/or smoking paraphernalia, in accordance with one embodiment of the present invention. Reference made to vaping and/or smoking paraphernalia herein may include, but is not limited to, a smoking pipe, a tobacco pipe, a bong, an electronic vaporizer, an e-cigarette, or other device particularly constructed and engineered for heating smokable or vaping material to produce a gaseous smoke or vapor for human consumption. As such, rock insert 10 is adapted and configured for use with any smoking or vaping paraphernalia having a designated port, opening or recess to accommodate receiving the rock insert 10 therein.

Rock insert 10 includes a porous body 12 having a sidewall 14, a closed bottom 16, and an interior or cavity 18 extending partially within the porous body 12 defining a top opening 20 including a top perimeter 22. In one embodiment, rock insert 10, and more particularly porous body 12 comprises an annular, or cylindrical body having a closed bottom 16 including a round or conical contour, as better illustrated in FIG. 2. It is appreciated that porous body 12 is formed and constructed from earth's natural resource material such as volcanic lava, extrusive and/or igneous substance or materials having a porous structure including a plurality of minute spaces, holes, pores, crevices, pockets, vesicles, cavities, and/or grain boundaries shown generally, for illustrative purposes, at P1, P2, and P3. The porous structure of the body 12 includes heat resistance properties that provides natural heat insulation properties that effectively absorbs heat, and evenly distributes heat when heated by an external heat source such as an open flame. The porous structure of the body 12 continues to deliver heat gradually over a prescribed period of time even after an external heat source is extinguished or removed. The heat insulating properties of the porous body 12 operates to shield and protect a liquid concentrate material or agent, retained within the interior 18 of the porous body 12 from burning quickly as a result of direct exposure to an open flame provided by an external heat source as often experienced in prior art devices. In some embodiments, the pores are interconnected and form porous networks through the porous body.

The porous body 12 is constructed from various types of materials including for example, but not limited to, volcanic rock or lava, extrusive and/or igneous materials, basalt, andesite, rhyolite, dacite, latite, pumice, pyroclastic rock, sandstone, siltstone, mudstone, shale, granite, Trachytic obsidian, Trachyte, Andesitic glass, Syenite, Granodiorite, Quartz diorite, Rhyolitic obsidian, various tuffite, clay, glass, fire rock, limestone, or any combination thereof. Red lava rock, technically called “scoria” provides a colorful addition to the rock insert 10 and comprises vesicular basaltic lava having very small pores or vesicles (<1 mm). Grey lava rock often referred to as “pumice” typically includes subtle layering of a volcanic glassy nature that comprises a mixture of rock and volcanic ash and includes an abundance of air thus being extremely light in weight. Red lava rock is generally denser in nature as it has less air infused into the rock as compared to pumice. In a preferred embodiment, the rock insert 10 is constructed using the more dense red lava rock.

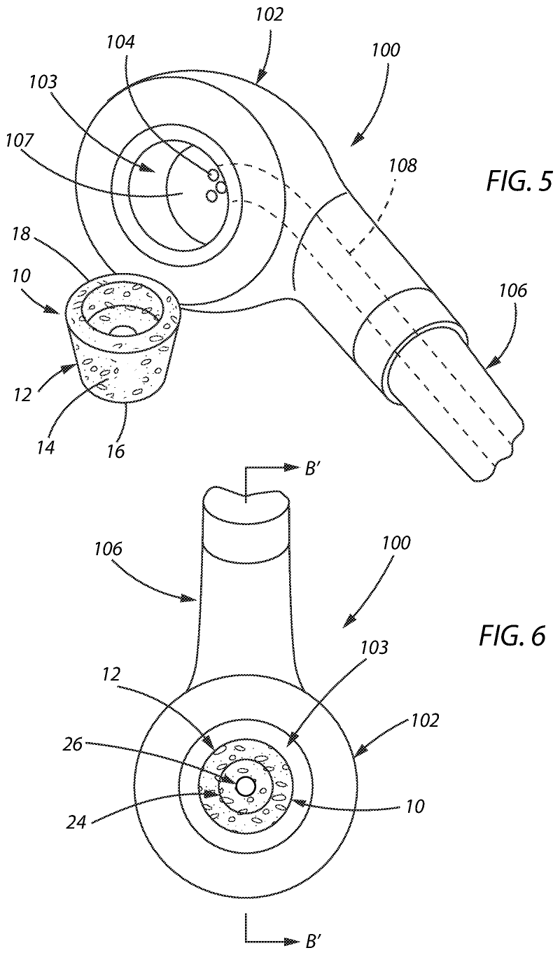

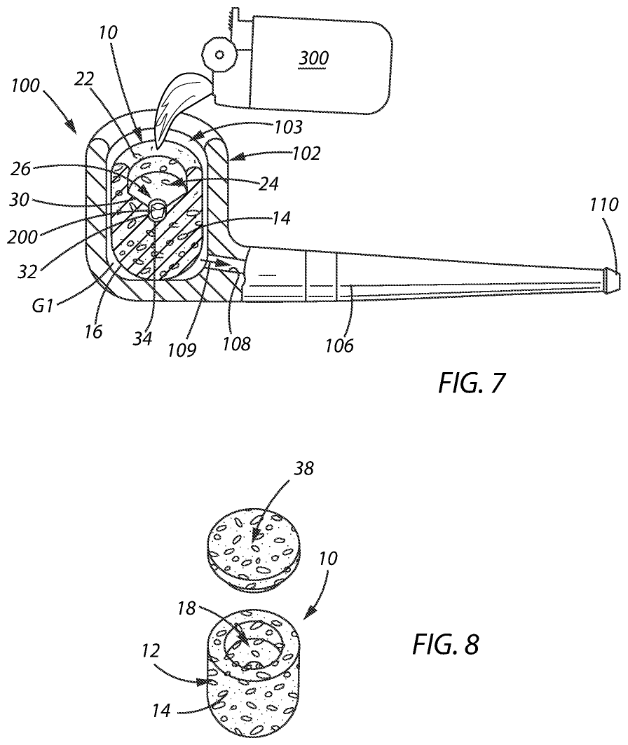

With reference made to FIG. 2, the rock insert 10 of FIG. 1 is shown inverted to illustrate the feature of the closed bottom 16 having a rounded contour, or a conical shape that is sized and shaped to facilitate inserting the rock insert 10 within a designated recess or opening of a smoking or vaping paraphernalia such as for example, a pipe bowl 102 of a smoking pipe 100, as better illustrated in FIG. 6. It is noted that the porous body 12 may comprise any shape and size produced by tooling machines or instruments to accommodate inserting the rock insert 10 within a designated recess or opening of a smoking or vaping paraphernalia device. For example, the rock insert 10 may be fabricated or constructed by a tumbling process to give it a desired shape. As such, the rock insert 10 may comprise any number of different sizes, diameters or shapes to accommodate use with various types of smoking or vaping paraphernalia. In one alternative example, the porous body 12 may comprise a square shape to accommodate fitting into a square recess of a smoking device. In the preferred embodiment, the rounded contour of the closed bottom 16 is configured to provide an air gap G1 for circulating air and promoting combustion while readily delivering gaseous vapor, under the influence of an inhaled force by a user, through intake openings 103 provided on the bottom surface of the pipe bowl 102, as better illustrated in FIGS. 5 and 7. In one exemplary embodiment, closed bottom 16 may include short, downward, extending feet or nubs for elevating the rock insert 10 upwards from the bottom surface of the bowl opening 104 to increase the size of the air gap G1 thereby increasing and enhancing the circulation of air, and smoke and/or vapor gases created when heating the smokable or vaping material.

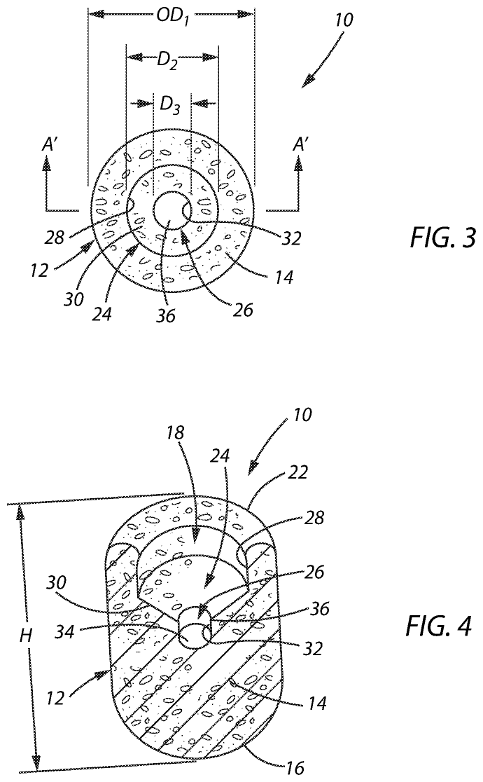

Turning now to FIGS. 3 and 4, there are shown a top view, and a perspective, cross-sectional view taken along the lines A′-A′ of FIG. 3, respectively, of rock insert 10, in accordance with one embodiment of the present invention. As described, rock insert 10 includes an interior 18 extending partially within the porous body 12 defining a first interior or cavity region 24 in communication with a second interior or cavity region 26. The first interior region 24 extends within the general, central area of the porous body 12 defining a first interior sidewall 28 integral with an annular base 30. The second interior region 26 extends through the annular base 30 and further into the porous body 12 defining a second sidewall 32 including a perimeter having a rim, an edge or sharp edge 36, and a closed base 34. In one exemplary embodiment, the first interior region 24 and the second interior region 26 comprise an annular or circular ledge 30 between them. In one exemplary embodiment, the first interior sidewall 28 comprises a generally vertical sidewall that is integral with a sloping or angled annular base or ledge 30. The sloping configuration of the annular base 30 helps retain the smokable or vaping material within the second interior region 26, and directs heat towards the smokable or vaping material as well. It is understood that the first interior sidewall 28 and/or the annular base or ledge 30 may comprise a same or different angle, or slope. For example, the first interior sidewall 28 and/or annular base 30 may comprise the same or a different angle, slope, orientation, configuration, or dimension, such as, for example, angled, beveled, diverging, converging, sloped, curved, planar, bulging, protruding, or including a convex and/or concave shape or comprise any combination thereof. In other embodiments, the first and second interior regions are not physically separated by an annular base or ledge 30, forming a continuous gradient from the top perimeter 22 to the closed base 34. For example, in one embodiment, there is a bump at the intersection between the first and second interior regions and in another embodiment, there is no change in the angle, slope, orientation, configuration, or dimension between the first and second interior regions where the first and second region include an integral sidewall to form a single interior.

In practice, rock insert 10 is shaped and sized for compatible use with a variety of smoking or vaping paraphernalia. For instance, in one non-limiting example, rock insert 10 is adapted for use with a smoking pipe 100 that generally includes a circular pipe bowl 102 having a bowl opening 104 for accommodating a smokable or vaping material. The rock insert 10 is generally shaped to compliment the shape of the bowl opening 104 of a smoking pipe 100. As noted, because smoking pipes may have differently sized bowl openings 104, the rock insert 10 may comprise a host of different sizes and shapes to accommodate different types of smoking pipes, or other smoking and/or vaping paraphernalia having designated ports, openings, or recesses of different sizes or shapes. In one exemplary embodiment, and as referenced in FIGS. 3 and 4, rock insert 10 includes a cylindrical porous body 12 having an outer diameter OD1 of 5 to 10 cm, with a preferred diameter of approximately 7 cm, and an overall height, denoted at H in FIG. 4, of 7 to 12 cm, with a preferred height of approximately 8 cm. The first interior region 24 may include a diameter denoted at D2 of 2 to 7 cm, with a preferred diameter D2 of approximately 4 cm, and a depth of 1 to 3 cm. The second interior region 26 may include a diameter denoted at D3 of 1 to 3 cm, with a preferred diameter D3 of approximately 2 cm, and a depth of 2 to 4 cm. It is noted that for exemplary purposes, the second interior region 26 has a smaller diameter than the diameter of the first interior region 24. The second interior region 26 is configured to receive smokable or vaping material therein in the form of a solid, semi-solid material, or a liquid concentrate. The second interior region 26 shelters smokable or vaping material from direct exposure to an open flame produced from an external heat source, such as a torch, match, or butane lighter. In circumstances where a solid or semi-solid concentrate material is employed, the porous body 12 absorbs heat produced by an external heating source, and gradually delivers the heat to the solid or semi-solid material to liquefy the concentrate to produce a gaseous vapor for human consumption. The porous structure of the second interior region 26 effectively absorbs and retains the liquefied concentrate or agent as its being heated. The small diameter of the second interior region 26 provides a narrow, shallow area for concentrate material to accumulate and absorb heat from the surrounding, porous structure of the body 12.

In another constructive embodiment, the first interior region 24 may include a diameter that is approximately 30% of the outside diameter OD1 of the porous body 12 of the rock insert 10. The depth of the first interior region 24 is approximately 10% of the total overall height H of the rock insert 10. The second interior region 26 is approximately 30% the diameter of the first interior region 24, and extends downwards approximately 20% of the total height H of the porous body 12 of the rock insert 10. It is understood that the rock insert 10 may be constructed from a natural resource, or a man-made resource using any number of tools, machines, or processes.

Turning now to FIGS. 5 and 6, there are shown a perspective view of the rock insert 10 readily adapted for use with a traditional smoking paraphernalia comprising, in one example, a smoking pipe 100. The traditional construction of a smoke pipe 100 typically includes a circular pipe bowl 102 including a bowl opening 103 having a plurality of intake openings 104 formed within the base 107 of the bowl opening 103, and a stem 106 conjoined to the pipe bowl 102. The stem 106 includes a gas delivery channel 108 (shown in dotted lines) that extends along an inner, central axis of the stem 106 and terminates at a mouth piece 110 provided at a distal end of the stem 106. The gas delivery channel 108 is in fluid communication with one or more intake openings 104 provided within the bowl opening 103. Gaseous smoke and/or vapor produced when heating a smokable or vaping material, flows within the intake openings 104 and passes through the gas delivery channel 108 and out from mouth piece 110 under inhaled force of a user.

In one embodiment, rock insert 10 is shaped and sized to correspondingly fit within the bowl opening 103 of the pipe bowl 102 under friction where the outer surface of sidewall 14 of the porous body 12 engages the inner surface of the bowl opening 103 preventing the rock insert 10 from easily falling out of the pipe bowl 102 during use. In one non-limiting embodiment, the porous body 12 may include engaging features formed integral with, or separately attached to, the outer surface of the sidewall 14 to help removably retain the rock insert 10 securely within the bowl opening 103. For example, such engaging features may include any of, embossed spirals, ridges, protrusions or nubs, or one or more bands or O-rings provided on the outer surface of sidewall 14 of the porous body 12. Types of material used for the engaging features may include fire and/or heat resistant metal, rubber, silicone, volcanic material, glass, ceramic, or fabric. For example, in one non-limiting embodiment, the rock insert 10 can include an o-ring formed integral with, or provided separately on, the outer surface of the sidewall 14 of the porous body 12 so that when the rock insert 10 is fully disposed within the bowl opening 103, the o-ring engages the inner surface of the bowl opening 104 securing the rock insert 10 in place.

With reference to FIG. 7, there is illustrated a side view of the smoke pipe 100 showing a perspective, cross-sectional view of the rock insert 10 fully seated within the pipe bowl 102 taken along lines B′-B′ of FIG. 6. For illustrative purposes, a smokable or vaping material comprising a solid, semi-solid, or liquid concentrate material, generally denoted at 200, is deposited within the interior 18 of the porous body 12 such that a portion of the deposited smokable material is received within the second interior region 26. The angled annular base 30 helps direct the smokable material towards entry of the second interior region 26. The second interior sidewall 32 and closed base 34, as illustrated in FIG. 4, help secure and retain the concentrate material 200 within the second interior region 26. In practice, an external heating source 300 is used to heat the porous body 12. In the representative example, the external heating source 300 comprises a butane lighter, however, other devices such as an electric heater, a match, candle, or a portable, hand-operated butane torch can be used. The butane lighter 300 is manipulated by a user to direct the open flame towards the upper region of the porous body 12 including the top perimeter 22, and an upper area of the first interior region 24 allowing the porous body 12 to absorb heat. The heat produced is captured within the multitude of pores vesicles formed within the porous body 12. The absorbed heat accumulates within the surrounding area and inwards of the second interior region 36 gradually heating the smokable or vaping material while preventing direct exposure to the open flame. The porous structure of the body 12 or rock insert 10 provides a flame resistant substrate that effectively absorbs heat, and delivers heat gradually to the smokable or vaping material 200 slowly in a controlled manner allowing the smokable or vaping material 200 to change states from a solid or semi-solid phase into a liquid concentrate. As the solid or semi-solid material 200 changes into a liquid concentrate, the liquid concentrate flows within the plurality of interconnected pores, crevices, pockets, vesicles, and grain boundaries of the porous structure of the rock insert 10 while absorbing heat evenly and slowly. As the flame from the lighter 300 is extinguished or removed, the liquid concentrate material 200 continues to absorb heat provided by the porous body 12. As the temperature continues to rise, the liquid concentrate material lodged in the plurality of interconnected pores, crevices, pockets, vesicles, and grain boundaries changes from a liquid state into a gaseous state releasing a gaseous vapor. The gaseous vapor passed through the intake openings 104 provided at the bottom surface 107 of the pipe bowl 102, and flows 109 through delivery channel 108 of stem 106, and out from mouthpiece 110 under inhaled force by a user for enjoyed consumption. As such, the solid or semi-solid material 200 is heated to a liquid concentrate, and then subsequently to a gaseous vapor in the presence and absence of a direct flame produced from an external heating source. The porous structure of the body 12 provides a flame resistant substrate that regulates the heat applied from the exposed flame, and allows the solid or semi-solid material 200 to change into a liquid concentrate and gaseous vapor without burning quickly. As some smokable or vaping materials, concentrates, or agents are sensitive to heat, continuously applying a direct flame would vaporize the heat sensitive liquefied concentrate too quickly compromising the ability to generate gaseous vapor. It is noted that users may employ a smokable or vaping material that comprises a liquid concentrate thus eliminating the need to transform a solid or semi-solid material into a liquid state.

Turning now to FIG. 8, there is shown a perspective view of the rock insert 10, and a cap 38 removably attachable to the top opening 20 of the porous body 12 for shielding smokable or vaping material deposited within the interior 18 of the rock insert, in accordance to an alternative embodiment of the present invention. The cap 38 is employed to shield the smokable or vaping material 200 from direct exposure of an open flame generated from an external heating source, and to protect the material from exposure. In a preferred embodiment, cap 38 is constructed from the same porous material used to construct rock insert 10. Cap 38 may include frictional engagement features to help retain the cap 38 to the top opening 20 of the porous body 12. The bottom surface of the cap 38 can be engineered to help direct heat downwards towards the first and second interior regions 24, 26 for heating smokable or vaping material disposed within the interior 18 of the porous body 12. For example, in one, non-limiting example, cap 38 may include a convex-shaped underside that extends within a portion of the first interior region 24 when attached to the top opening 20 of the rock insert 10. The downward convex projection directs heat downwards towards the smoking or vaping material retained within the second interior region 26.

Various changes, alterations, and modifications can be made to the rock insert 10 to improve or enhance performance, or to provide additional advantages of operation and use. For example rock insert 10 may comprise any color, and density, porosity, and permeability sufficiently necessary to permit a liquid concentrate to enter the porous structure and absorb heat gradually to change the liquid concentrate from a liquid state into a gaseous vapor in a controlled manner for human inhaled consumption. It is understood that the size, shape, configuration, and dimensions OD1, D2, D3, H of the body 12 or rock insert 10 may be altered, modified, or changed to accommodate use of the rock insert 10 with various traditional and/or specifically designed smoking and/or vaping paraphernalia. The overall height H of the porous body 12 may be slightly increased to extend slightly higher than the surrounding upper perimeter or rim of the pipe bowl 102 to provide an area for users to grasp the rock insert 10 with their fingers when inserting and removing the rock insert 10 from a designated paraphernalia recess, opening or bowl. In addition, it is understood that although the rock insert 10 is shown to include a cylindrical porous body 12, it is understood that the porous body 12 may comprise other geometric shapes, sizes, and configurations to accommodate use with various smoking pipes, and vaping devices. As such, the porous body 12 may comprise any of an oblong, oval, ellipse, square, rectangular, or semi-circular shape.

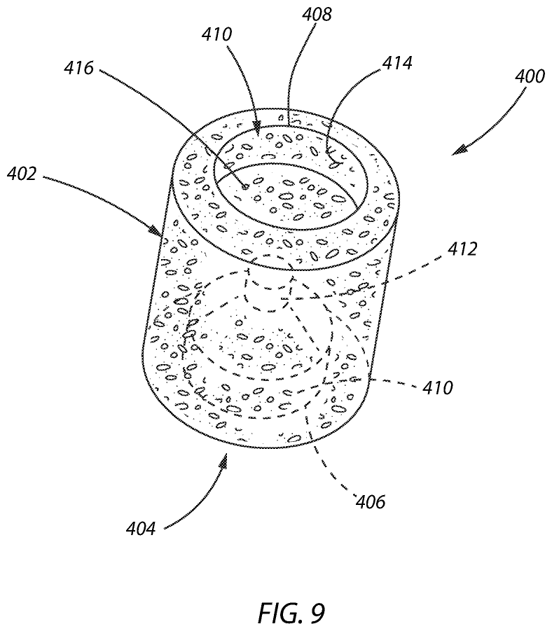

In reference to FIG. 9, there is illustrated a perspective view of a rock insert 400 showing a cylindrical porous body 402 including an interior 404 extending within one end of the porous body 402 for receiving a smokable or vaping material, a top opening 406 in communication with the interior 404, a bottom 408, and an alternate interior 410 formed within the bottom 408, opposite interior 404, in accordance with an alternative embodiment of the present invention. Rock insert 400 is similar in construction and operation to rock insert 10 with the exception of the inclusion of an alternate interior 410 formed at the opposite end of interior 404. As described, rock insert 400 includes an interior 404 extending within the cylindrical porous body 402 and including a first interior region 410 in communication with a second interior region 412 similar to interior regions 24, 26 of porous body 12. An alternate interior 410 is formed within bottom 408 of the porous body 402 defining an alternate sidewall 414 integral with an alternate closed base 416. The alternate interior 410 may include a single interior or in one embodiment, include an alternate first and second interior region (not shown) similar to the first and second interior regions 24, 26 of porous body 12 of rock insert 10. The alternate interior 410 may comprise any diameter, depth, and shape. Rock insert 400 provides multiple interiors 404, 410 to accommodate use with a variety of different smokable or vaping materials. In practice, users can removably insert the rock insert 400 into a pipe bowl 102 of a smoking pipe 100 to expose interior 404 for use with a certain type of smokable or vaping material, or alternatively, users can invert and reposition the rock insert 400 into the pipe bowl 102 to expose the alternate interior 410 to accommodate use with a different type of smokable or vaping material.

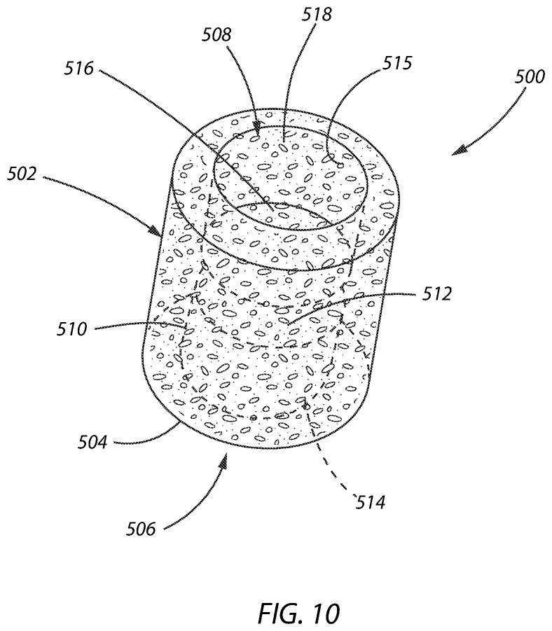

There is shown in FIG. 10, a side, perspective view of a rock insert 500 including a porous body 502 having a first interior 506 formed in one end 504, and a second interior 508 formed in another end, opposite the one end 504, in accordance with an alternative embodiment of the present invention. The porous body 502 may comprise any shape and size, but in a preferred embodiment, comprises a cylindrical shape. The first interior 506 extends within a general, central area of one end 504 of the porous body 502 defining a first interior sidewall 510 integral with a first closed base 512, and a first opening 514. The second interior 508 extends within a general, central area of another end of the porous body 502, opposite the one end 504, defining a second interior sidewall 515 integral with a second closed base 516, and a second opening 518. In one embodiment, the first and second sidewall 510, 515 may each taper outwards, or extend vertically when extending from respective closed base 512, 516 towards openings 514, and 518. Alternatively, the first sidewall 510 may taper inwards, while the second sidewall 515 extends vertically as the sidewalls 510, 515 extend respectively from each closed base 512, 516 towards openings 514, 518, or vice-versa. It is noted that each closed base 512, 516 is distinctly separated apart from each other a predefined distance. The first and second interior 506, 508 may each comprise the same or different dimension, size and/or shape. For example, in one embodiment, the first interior 506 may be equal in dimension, size, and/or shape to that of the second interior 508 where each interior 506, 608 holds the same amount of smokable and/or vaping material therein. In yet another embodiment, the first interior 506 may comprise a smaller depth than that of the second interior 508. It is understood that inclusion of the first and second interior 506, 508 provides a functional benefit. For example, as the rock insert 500 is used over time, any excess smokable or vaping materials, such as resin or liquid concentrate, that flows downwards towards the bottom end of the rock insert 500 when retained within a designated opening or recess of a smoking or vaping paraphernalia becomes accessible when the rock 500 is inverted for use. Any lingering resin or liquid concentrate that flowed towards the bottom is simply reheated to produce a gaseous vapor for inhaled consumption.

Since many modifications, variations, and changes in detail can be made to the described preferred embodiments of the invention, it is intended that all matters in the foregoing description and shown in the accompanying drawings be interpreted as illustrative and not deemed to be limiting. Furthermore, it is understood that any of the features presented in the embodiments may be integrated into any of the other embodiments unless explicitly stated otherwise. The scope of the invention should be determined by the appended claims and their legal equivalents. Modifications, additions, or omissions may be made to the systems, apparatuses, and methods described herein without departing from the scope of the disclosure. For example, the components of the system may be integrated together, or separated. Moreover, the operations of the systems and apparatuses disclosed herein may be performed by more, fewer, or other components and the methods described may include more, fewer, or other steps.

What is claimed is:

1. A rock insert for smoking or vaping, said rock insert comprising:
a porous body including an interior extending partially within a central region of said porous body, a top opening in communication with said interior, and a closed bottom, said interior including a first interior region having a first interior sidewall integral with an annular base, and a second interior region formed through said annular base and coincident with said first interior region, said second interior region including a second interior sidewall having a top peripheral edge and a closed base, said second interior region being smaller in size than said first interior region; and
wherein said porous body is heatable by an external heating source to gradually distribute heat to a smokable or vaping material deposited within said interior to produce a gaseous vapor for human consumption.

2. The rock insert of claim 1, wherein said porous body comprises a cylindrical porous body having an outer diameter and height.

3. The rock insert of claim 2, wherein said first interior region comprises a first diameter, and said second interior region comprises a second diameter, said second diameter being smaller than said first diameter.

4. The rock insert of claim 3, wherein said closed bottom comprises a rounded or conical contour.

5. The rock insert of claim 1, wherein the porous body comprises a microporous, macroporous, cryptoporous, ultramicroporous, or mesoporous material.

6. The rock insert of claim 5, wherein said porous body comprises any of volcanic rock, lava, scoria, basalt, andesite, rhyolite, dacite, latite, pumice, pyroclastic rock, sandstone, siltstone, mudstone, shale, granite, Trachytic obsidian, Trachyte, Andesitic glass, Syenite, Granodiorite, Quartz diorite, Rhyolitic obsidian, various tuffite, clay, glass, firerock, limestone, or any combination thereof.

7. The rock insert of claim 6, wherein said first interior sidewall comprises a vertical wall integral with said annular base, said annular base sloping downwards towards an opening of said second interior region.

8. The rock insert of claim 5, further including a porous cap removably attachable to said top opening of said porous body for closing said interior, said porous cap comprising a microporous, macroporous, cryptoporous, ultramicroporous, or mesoporous material.

9. The rock insert of claim 1, further including an alternate interior formed within said closed bottom and including an alternative sidewall integral with an alternate closed base, said alternate interior formed opposite said interior.

10. A smoking or vaping accessory comprising:
a cylindrical porous body including an interior extending within said porous body, a top opening in communication with said interior, and a closed bottom having a round contour, said interior including a first interior region having a first interior sidewall integral with an annular ledge, and a second interior region formed coincident with said first interior region, and including a second interior sidewall and a closed base, said first interior region comprising a first diameter, and said second interior region comprising a second diameter, said second diameter being smaller than said first diameter; and
wherein said cylindrical porous body is heatable by an external heating source to gradually distribute heat to a smokable or vaping material provided within said interior to produce a gaseous vapor for human consumption.

11. The smoking or vaping accessory of claim 10, wherein the cylindrical porous body comprises a microporous, macroporous, cryptoporous, ultramicroporous, or mesoporous material.

12. The smoking or vaping accessory of claim 11, wherein said cylindrical porous body comprises any of volcanic rock, lava, scoria, basalt, andesite, rhyolite, dacite, latite, pumice, pyroclastic rock, sandstone, siltstone, mudstone, shale, granite, Trachytic obsidian, Trachyte, Andesitic glass, Syenite, Granodiorite, Quartz diorite, Rhyolitic obsidian, various tuffite, clay, glass, firerock, limestone, or any combination thereof.

13. The smoking or vaping accessory of claim 12, wherein said cylindrical porous body includes an outer diameter of 5 to 10 cm, and a height of 7 to 12 cm, said first interior region includes a diameter of 4 to 7 cm, and a depth of 1 to 3 cm, and said second interior region includes a diameter of 1 to 3 cm, and a depth of 2 to 4 cm.

14. The smoking or vaping accessory of claim 12, further including an alternate interior formed within said closed bottom and including an alternate sidewall integral with an alternate closed base, said alternate interior formed opposite said interior.

15. A volcanic rock removably seated in a smoking or vaping paraphernalia, said volcanic rock comprising:
a porous body including a first interior extending partially within one end of said porous body, said first interior including a first interior sidewall integral with a first closed base, and a first opening, and a second interior extending partially within another end of said porous body, opposite said one end, said second interior including a second interior sidewall integral with a second closed base, and a second opening, said first interior and a said second interior aligned one on top of another along a vertical axis when said porous body is oriented vertically; and
wherein said porous body is heatable by an external heating source to gradually distribute heat to a smokable or vaping material held within either of said first or second interior of said porous body to produce a gaseous vapor for human consumption.

16. The volcanic rock of claim 15, wherein said porous body comprises a microporous, macroporous, cryptoporous, ultramicroporous, or mesoporous body.

17. The volcanic rock of claim 16, wherein said porous body includes a plurality of pores and/or vesicles having different sizes, shapes, and depths that are formed randomly within the porous body.

18. The volcanic rock of claim 17, wherein said porous body is constructed from scoria or pumice.

19. The volcanic rock of claim 1, wherein said first interior and said second second interior each comprise a same shape and dimension.

20. The volcanic rock of claim 18, wherein said porous body comprises a cylindrical porous body.
